(19) United States
(12) Patent Application Publication
(10) Pub. No.: US 2008/0056987 A1
(43) Pub. Date: Mar. 6, 2008

(54) Quinoline derivatives, their preparation, their use, and medicaments comprising them

(76) Inventors: Olaf Prien, Berlin (DE); Knut Eis, Berlin (DE); Duy Nguyen, Berlin (DE); Christoph Huwe, Berlin (DE); Wolfgang Schwede, Glienicke (DE); Judith Guenther, Berlin (DE); Dieter Zopf, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/765,683

(22) Filed: Jun. 20, 2007

Related U.S. Application Data
(60) Provisional application No. 60/816,631, filed on Jun. 27, 2006.

(30) Foreign Application Priority Data
Jun. 21, 2006 (DE) 10 2006 0294 46.7

(51) Int. Cl. A61K 51/00; A61K 31/47; A61P 25/00; A61P 9/00; C07D 295/00; C12Q 1/00; C12N 9/99; C07D 215/00; A61P 43/00; A61K 31/5377

(52) U.S. Cl. 424/1.11; 435/184; 435/4; 514/232.8; 514/291; 514/293; 544/126; 546/80; 546/83

(57) ABSTRACT

The present invention relates to a quinoline derivative having the general formula (A) 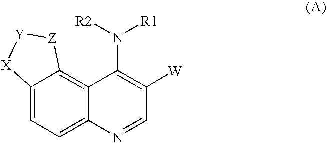
in which R¹, R², W, X, Y and Z are indicated in the description and the claims, the use of the compounds of the general formula (A) for the treatment of various disorders, and the preparation of compounds of the general formula (A).

This application claims benefit of the filing date of U.S. Provisional Application Ser. No. 60/816,631 filed, Jun. 27, 2006, which is incorporated by reference herein.

The invention relates to certain quinoline derivatives, their preparation and use as inhibitor of protein kinases, in particular of Eph (erythropoetin-producing hepatoma amplified sequence) receptors for the treatment of various disorders.

Protein tyrosine kinases catalyze the phosphorylation of specific tyrosine residues in various proteins. Such phosphorylation reactions play a part in a large number of cellular processes which are involved in the regulation of growth and differentiation of cells. Protein tyrosine kinases are divided into receptor and non-receptor tyrosine kinases. The family of receptor tyrosine kinases (RTKs) consists of 58 kinases (Manning G. et al. 2002, Science 298, 1912-1934). RTKs have an extracellular ligand binding domain, a transmembrane domain and an intracellular domain which usually comprises the tyrosine kinase activity. RTKs mediate signal transduction from extracellular stimulators such as, for example, growth factors. The ligand binding leads to dimerization of the RTKs and reciprocal autophosphorylation of their intracellular domains. Depending on the cell type, specific intracellular binding proteins are recruited thereby (inter alia non-receptor tyrosine kinases), via which signal processing takes place in the cell (Schlessinger J. 2000, Cell 103, 211-225). These include receptor families of growth factors such as EGF (epidermal growth factor), VEGF (vascular endothelial growth factor), FGF (fibroblast growth factor), PDGF (platelet derived growth factor) and NGF (nerve growth factor), and of the insulin receptors, and the large family of ephrin receptors and others.

The ephrin (Eph) receptors constitute the largest family within the RTKs. They are divided according to their sequential relationship and their ligand specificity into the group of EphA receptors (9 members) and of EphB receptors (6 members) (Kullander K. and Klein R. 2002, Nat. Rev. Mol. Cell Biol. 3, 475-486; Cheng N. et al. 2002, Cyt. and growth factor Rev. 13, 75-85.). Eph receptors are activated by membrane-associated ligands of the EphrinA or EphrinB family. EphrinAs are anchored in the cell membrane via glycolipids (GPI), whereas EphrinBs have a transmembrane region and an intracellular domain. The interaction between Ephrins and the Eph receptors leads to a bidirectional signal transmission in the ephrin-expressing and in the Eph-receptor-carrying cells. Ephrins and Eph receptors play a part in a large number of morphogenetic processes in embryonic development and in the adult organism. They are involved in embryo patterning, in the development of the blood vessel system (Gerety S. S: et al 1999, Mol. Cell 4, 403-414) and in the establishment of neuronal interconnections (Flanagan, J. G. and Vanderhaeghen, P., 1998, Annu. Rev. Neurosci. 21, 309-345). In the adult organism, they are involved in neovascularization processes, e.g. in tumour development and in endometriosis, and in the morphogenesis of the intestinal epithelium (Batlle E. et al. 2002, Cell 111:251-63.). At the cellular level, they mediate migration, adhesion and juxtacrine cell contacts. Elevated expression of Eph receptors such as, for example, EphB2 and EphB4 has also been observed in various tumour tissues such as, for example, breast and bowel tumours (Nakamoto M. and Bergemann A. D. 2002, Mic. Res. Tech. 59, 58-67). EphB2, EphB3 and EphB4 knockout mice show defects in the formation of the blood vessel system. The embryonic lethality of EphB4−/− mice in embryonic stage d14 shows the special role of EphB4 in this process (Gerety S. S: et al 1999, Mol. Cell 4, 403-414). Modulation of these receptors, e.g. by inhibiting their kinase activity, leads for example to suppression of tumour growth and/or tumour metastasis either through a direct antitumour or through an indirect antiangiogenic effect.

Non-receptor tyrosine kinases occur in soluble form inside cells and are involved in the processing of extracellular signals (e.g. from growth factors, cytokines, antibodies, adhesion molecules) inside the cell. They include inter alia the families of src (sarcoma) kinases, of Tec (tyrosine kinase expressed in hepatocellular carcinoma) kinases, of Abl (Abelson) kinases and of Brk (breast tumor kinase) kinases, and the focal adhesion kinase (FAK).

An altered activity of these protein tyrosine kinases may lead to a wide variety of physiological disorders in the human body and thus cause for example inflammatory, neurological and oncological disorders.

WO 01/19828 A discloses a wide variety of kinase inhibitors.

US 2004116388 A discloses triazine compounds which inhibit receptor tyrosine kinases.

WO 03/089434 A discloses imidazo[1,2a]pyrazin-8-ylamines, and WO 04/00820 A discloses various aromatic monocycles, which inhibit receptor tyrosine kinases.

EP 0 187 705 A2 describes imidazo[4,5f]quinolines which exhibit an immunomodulating effect in infectious diseases. Likewise, U.S. Pat. No. 5,506,235 A describes imidazo[4,5f]quinolines with an immunostimulating effect.

WO 04/006846 A discloses various quinazoline derivatives which inhibit receptor tyrosine kinases.

WO 03/053960 describes substituted 3-cyanoquinoline derivatives as MEK inhibitors.

US 2005/0026933 claims quinolinecarbonitriles as EGFR inhibitors.

WO 01/68186 describes cyanoquinolines for the treatment of intestinal polyps.

However, no Eph receptor inhibitors are described among the receptor tyrosine kinase inhibitors.

It is an object of the present invention to provide compounds which inhibit receptor tyrosine kinases, especially Eph receptors.

The object is achieved by quinoline derivatives having the general formula (A), a process for preparing the quinoline derivative, the uses of the quinoline derivative, and a medicament comprising the quinoline derivative, according to the following description and the claims.

The present invention relates to a quinoline derivative having the general formula (A): 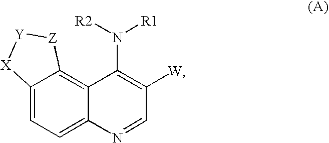
where
W is equal to methyl, C(O)OR⁴, C(O)NR³R⁴;
R¹ and R² are identical or different and are selected independently of one another from the group comprising hydrogen, hydroxy, halogen, nitro, cyano, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚPO₃(R⁶)₂, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵, or —OR⁵, where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally form together a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
   the N-oxides, solvates, hydrates, stereoisomers, diastereomers, enantiomers and salts thereof.

A preferred subgroup are compounds in which:
W is equal to methyl, C(O)OR⁴, C(O)NR³R⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₋₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
   the N-oxides, solvates, hydrates, stereoisomers, diastereomers, enantiomers and salts thereof.

In a variation, quinoline derivatives of the general formula (A) are claimed, where:
W is equal to C(O)OR⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, 15-(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
   the N-oxides, solvates, hydrates, stereoisomers, diastereomers, enantiomers and salts thereof.

More preference is given to compounds of the general formulae (A1) to (A5): 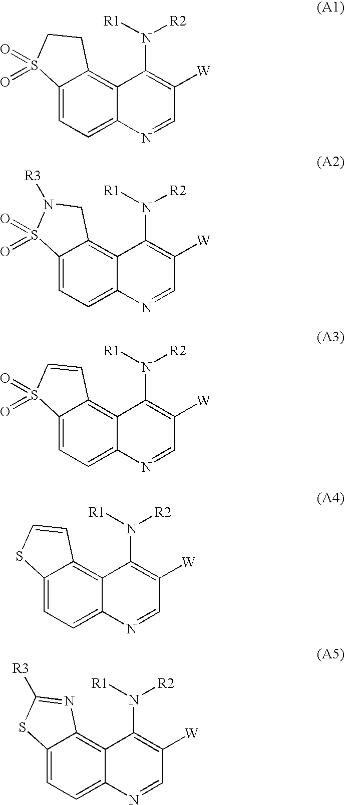
where:
W is equal to methyl, C(O)OR⁴, C(O)NR³R⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
   the N-oxides, solvates, hydrates, stereoisomers, diastereomers, enantiomers and salts thereof.

Particular preference is given to compounds of the general formulae: 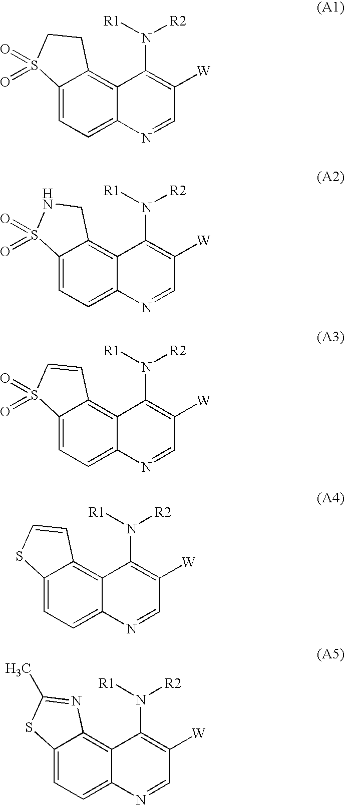
where:
W is equal to methyl, C(O)OR⁴, C(O)NHR⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
R⁴ is hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
   the N-oxides, solvates, hydrates, stereoisomers, diastereomers, enantiomers and salts thereof.

The following compounds are preferred
ethyl 9-(2-hydroxyethylamino)-3,3-dioxo-2,3-dihydro-1H-3λ⁶-thieno[3,2-f]quinoline-8-carboxylate;
ethyl 9-(3-hydroxypropylamino)-3,3-dioxo-2,3-dihydro-1H-3λ⁶-thieno[3,2-f]-quinoline-8-carboxylate;
ethyl 9-(2-dimethylaminoethylamino)-3,3-dioxo-2,3-dihydro-1H-3λ⁶-thieno[3,2-f]quinoline-8-carboxylate;
ethyl 9-(2-acetylaminoethylamino)-3,3-dioxo-2,3-dihydro-1H-3λ⁶-thieno[3,2-f]quinoline-8-carboxylate;
ethyl 9-(2-morpholin-4-ylethylamino)-3,3-dioxo-2,3-dihydro-1H-3λ⁶-thieno[3,2-f]-quinoline-8-carboxylate;
ethyl 9-cyclopropylamino-3,3-dioxo-2,3-dihydro-1H-3λ⁶-thieno[3,2-f]quinoline-8-carboxylate;
ethyl 9-isopropylamino-3,3-dioxo-2,3-dihydro-1H-3λ⁶-thieno[3,2-f]quinoline-8-carboxylate;
ethyl 9-benzylamino-3,3-dioxo-2,3-dihydro-1H-3λ⁶-thieno[3,2-f]quinoline-8-carboxylate;
ethyl 3,3-dioxo-9-phenylamino-2,3-dihydro-1H-3λ⁶-thieno[3,2-f]quinoline-8-carboxylate;
ethyl 9-(4-methoxyphenylamino)-3,3-dioxo-2,3-dihydro-1H-3λ⁶-thieno[3,2-f]-quinoline-8-carboxylate;
ethyl 9-(4-hydroxyphenylamino)-3,3-dioxo-2,3-dihydro-1H-3λ⁶-thieno[3,2-f]-quinoline-8-carboxylate;
ethyl 9-phenylaminothieno[3,2-f]quinoline-8-carboxylate;
ethyl 9-benzylaminothieno[3,2-f]quinoline-8-carboxylate.

It has been found that the compounds according to the invention are able to inhibit receptor tyrosine kinases, especially Eph receptors.

Alkyl means in each case a straight-chain or branched alkyl radical such as, for example, methyl, ethyl, propyl, isopropyl, butyl, isobutyl, sec-butyl, tert-butyl, pentyl, isopentyl, hexyl, heptyl, octyl, nonyl and decyl.

Alkoxy means in each case a straight-chain or branched alkoxy radical such as, for example, methyloxy, ethyloxy, propyloxy, isopropyloxy, butyloxy, isobutyloxy, sec butyloxy, pentyloxy, isopentyloxy, hexyloxy, heptyloxy, octyloxy, nonyloxy or decyloxy.

The alkenyl substituents are in each case straight-chain or branched, with the following radicals being meant for example: vinyl, propen-1-yl, propen-2-yl, but-1-en-1-yl, but-1-en-2-yl, but-2-en-1-yl, but-2-en-2-yl, 2-methylprop-2-en-1-yl, 2-methylprop-1-en-1-yl, but-1-en-3-yl, but-3-en-1-yl, allyl.

Alkynyl means in each case a straight-chain or branched alkynyl radical which comprises two to six, preferably two to four, C atoms. Examples of suitable radicals are the following: ethynyl, propyn-1-yl, propyn-3-yl, but-1-yn-1-yl, but-1-yn-4-yl, but-2-yn-1-yl, but-1-yn-3-yl.

Cycloalkyl means monocyclic alkyl rings such as cyclopropyl, cyclobutyl, cyclopentyl, cyclohexyl or cycloheptyl, but also bicyclic rings or tricyclic rings such as, for example, adamantanyl. The cycloalkyl rings may be unsubstituted or substituted one or more times. Cycloalkyls according to this invention comprise C₃-C₁₂ carbon atoms; cycloalkyls having C₃-C₁₀ carbon atoms are preferred, and cycloalkyls having C₃-C₆ carbon atoms are particularly preferred.

An aryl radical has 6-12 carbon atoms in each case. The radical may be mono- or bicyclic, for example naphthyl, biphenyl and, in particular, phenyl.

The heteroaryl radical includes an aromatic ring system which comprises in each case 5-18 ring atoms, preferably 5 to 10 ring atoms and particularly preferably 5 to 7 ring atoms and, instead of the carbon, one or more identical or different heteroatoms from the group of oxygen, nitrogen or sulphur. The radical may be mono-, bi- or tricyclic and additionally in each case benzo-fused. However, only those combinations which are sensible in the view of a skilled person, especially in relation to the ring tension, are meant.

The heteroaryl rings may be unsubstituted or substituted one or more times. Examples which may be mentioned are: thienyl, furanyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, triazinyl and benzo derivatives of these radicals such as, for example, 1,3-benzodioxolyl, benzofuranyl, benzothienyl, benzoxazolyl, benzimidazolyl, indazolyl, indolyl, isoindolyl, oxepinyl, azocinyl, indolizinyl, indolyl, isoindolyl, indazolyl, benzimidazolyl, purinyl, quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl etc.

Halogen means in each case fluorine, chlorine, bromine or iodine.

C₃-C₁₂-Heterocycloalkyl stands for an alkyl ring including 3-12 carbon atoms, preferably including 3 to 10 carbon atoms and particularly preferably including 3 to 6 carbon atoms, which is interrupted by at least one of the following atoms nitrogen, oxygen and/or sulphur in the ring and which may optionally be interrupted by one or more identical or different —(CO)—, —SO— or —SO₂— groups in the ring and optionally comprises one or more double bonds in the ring. However, only those combinations which are sensible in the view of a skilled person, especially in relation to the ring tension, are meant. C₃-C₁₂-Heterocycloalkyls according to this invention are monocyclic, but also bicyclic or tricyclic. Examples of monocyclic heterocyclyles which may be mentioned are: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, trithianyl, quinuclidinyl etc.

As used in this application, “C₁-C₁₀” refers, for example in connection with the definition of “C₁-C₁₀-alkyl”, to an alkyl group having a finite number of 1 to 10 carbon atoms, i.e. 1, 2, 3, 4, 5, 6, 7, 8, 9 or 10 carbon atoms. The definition of “C₁-C₁₀” is further interpreted to mean that every possible sub-range such as, for example, C₁-C₁₀, C₂-C₉, C₃-C₈, C₄-C₇, C₅-C₆, C₁-C₂, C₁-C₃, C₁-C₄, C₁-C₅, C₁-C₆, C₁-C₇, C₁-C₈, C₁-C₉, C₁-C₁₀, preferably C₁-C₂, C₁-C₃, C₁-C₄, C₁-C₅, C₁-C₆; preferably C₁-C₄ is also included in the definition.

In analogy thereto “C₂-C₁₀” refers, for example in connection with the definition of “C₂-C₁₀-alkenyl” and “C₂-C₁₀-alkynyl”, to an alkenyl group or alkynyl group having a finite number of 2 to 10 carbon atoms, i.e. 2, 3, 4, 5, 6, 7, 8, 9 or 10 carbon atoms. The definition of “C₂-C₁₀” is interpreted to mean that every possible sub-range such as, for example, C₂-C₁₀, C₃-C₉, C₄-C₈, C₅-C₇, C₂-C₃, C₂-C₄, C₂-C₅, C₂-C₆, C₂-C₇, C₂-C₈, C₂-C₉, preferably C₂-C₄, is also included in the definition.

Furthermore, “C₁-C₆” refers, for example in connection with the definition of “C₁-C₆-alkoxy” to an alkoxy group having a finite number of 1 to 6 carbon atoms, i.e. 1, 2, 3, 4, 5 or 6 carbon atoms. The definition of “C₁-C₆” is interpreted to mean that every possible sub-range such as, for example, C₁-C₆, C₂-C₅, C₃-C₄, C₁-C₂, C₁-C₃, C₁-C₄, C₁-C₅, C₁-C₆; preferably C₁-C₄, is also included in the definition.

All statements of ranges in the application which are not explicitly mentioned here are defined analogously like the ranges “C₁-C₁₀”, “C₂-C₁₀” and “C₁-C₆” mentioned above as examples.

The term “one or more times”, e.g. in the definition of the substituents of the compounds of the general formulae of the present invention, is understood as meaning “one, two, three, four or five times, particularly one, two, three or four tines, more particularly one, two or three times, more particularly one or two times”.

Isomers mean chemical compounds of the same molecular formula but different chemical structure. A distinction is made in general between constitutional isomers and stereoisomers. Constitutional isomers have the same molecular formula but differ through the mode of linkage of their atoms or atomic groups. Included herein are functional isomers, positional isomers, tautomers or valence isomers. Stereoisomers have fundamentally the same structure (constitution) and thus also the same molecular formula, but differ through the spatial arrangement of the atoms. In general, configurational isomers and conformational isomers are distinguished. Configurational isomers are stereoisomers which can be interconverted only by breaking bonds. These include enantiomers, diastereomers and E/Z (cis/trans) isomers. Enantiomers are stereoisomers which are related to one another as image and mirror image and have no plane of symmetry. All stereoisomers which are not enantiomers are referred to as diastereomers. E/Z (cis/trans) isomers at double bonds are a special case. Conformational isomers are stereoisomers which can be interconverted by rotation of single bonds. To distinguish the types of isomerism from one another, see also the IUPAC rules section E (Pure Appl. Chem. 1976, 45, 11-30).

The quinoline derivatives according to the invention having the general formula (A) also encompass the possible tautomeric forms and include the E or Z isomers or, if a chiral centre is present, also the racemates and enantiomers. By these are also meant double-bond isomers.

The quinoline derivatives according to the invention may also exist in the form of solvates, in particular of hydrates, in which case the compounds according to the invention accordingly comprise polar solvents, in particular water, as structural element of the crystal lattice of the compounds according to the invention. The proportion of polar solvent, in particular water, may be in a stoichiometric or else non-stoichiometric ratio. Terms used in connection with stoichiometric solvates, hydrates are also hemi-, (semi-), mono-, sesqui-, di-, tri-, tetra-, penta-, etc. solvates or hydrates.

N-Oxides means that at least one nitrogen of the compounds according to the invention of the general formula (A) may be oxidized.

If an acidic function is present, suitable salts are the physiologically tolerated salts of organic and inorganic bases such as, for example, the readily soluble alkali metal and alkaline earth metal salts, and salts of N-methylglucamine, dimethylglucamine, ethylglucamine, lysine, 1,6-hexanediamine, ethanolamine, glucosamine, sarcosine, serinol, trishydroxymethylamino-methane, aminopropanediol, Sovak base, 1-amino-2,3,4-butanetriol.

If a basic function is present, the physiologically tolerated salts of organic and inorganic acids are suitable, such as hydrochloric acid, hydrobromic acid, sulphuric acid, phosphoric acid, oxalic acid, malonic acid, maleic acid, citric acid, succinic acid, tartaric acid and others.

Functional groups may be protected where appropriate by protective groups during the reaction sequence. Such protective groups may be inter alia esters, amides, ketals/acetals, nitro groups, carbamates, alkyl ethers, allyl ethers, benzyl ethers or silyl ethers. Compounds which may occur as constituent of silyl ethers inter alia are such as, for example, trimethylsilyl (TMS), tert-butyldimethylsilyl (TBDMS), tert-butyldiphenylsilyl (TBDPS), triethylsilyl (TES), etc. The preparation thereof is described in the experimental section.

The quinoline derivatives according to the invention having the general formula (A) inhibit receptor tyrosine kinases, especially Eph kinases, on which their effect is also based, for example in the treatment of disorders in which angiogenesis, lymphangiogenesis or vasculogenesis are involved, of disorders of the blood vessels, disorders caused by hyperproliferation of body cells, or chronic or acute neurodegenerative disorders. The present quinoline derivatives having the general formula (A) can accordingly be used as medicaments.

Treatments are preferably carried out on humans, but also on related mammalian species such as, for example, dog and cat.

Angiogenic and/or vasculogenic disorders can be treated by the growth of blood vessels being inhibited (antiangiogenic) or promoted (proangiogenic). Antiangiogenic uses take place for example in tumour angiogenesis, endometriosis, in diabetes-related or other retinopathies or in age-related macular degeneration. Proangiogenic uses take place for example in myocardial infarction or acute neurodegenerative disorders due to ischaemias of the brain or neurotraumata.

Blood vessel disorders mean stenoses, arterioscleroses, restenosis or inflammatory diseases such as rheumatoid arthritis.

Hyperproliferative disorders mean solid tumours, non-solid tumours or non-carcinogenic hyperproliferation of cells in the skin, where solid tumours mean inter alia tumours of the breast, colon, kidney, lung and/or brain. Non-solid tumours mean inter alia leukaemias, and non-carcinogenic hyperproliferation of cells in the skin means inter alia psoriasis, eczemas, scleroderma or benign prostatic hypertrophy.

Chronic neurodegenerative disorders mean inter alia Huntington's disease, amyotrophic lateral sclerosis, Parkinson's disease, AIDS-induced dementia or Alzheimer's disease.

The quinoline derivatives having the general formula (A) can likewise be used for diagnostic purposes in vitro or in vivo for identifying receptors in tissues by means of autoradiography and/or PET.

The substances can in particular for diagnostic purposes also be radiolabelled.

For use of the quinoline derivatives according to the invention as medicaments, they are converted into the form of a pharmaceutical product which, besides the active ingredient, comprises pharmaceutical, organic or inorganic inert carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules or in liquid form, for example as solutions, suspensions or emulsions. They additionally comprise where appropriate excipients such as preservatives, stabilizers, wetting agents or emulsifiers; salts to modify the osmotic pressure or buffers.

The present invention likewise relates to these pharmaceutical products.

Suitable for parenteral use are in particular solutions for injection or suspensions, especially aqueous solutions of the active compounds in polyhydroxyethoxylated castor oil.

Carrier systems which can also be used are surface-active excipients such as salts of bile acids or animal or vegetable phospholipids, but also mixtures thereof, and liposomes or their constituents.

Suitable for oral use are, in particular, tablets, coated tablets or capsules with talc and/or hydrocarbon carriers or binders, such as, for example, lactose, maize starch or potato starch. Use can also take place in liquid form, for example as solution, to which a sweetener is added where appropriate.

The present invention likewise relates to the enteral, parenteral and oral administrations.

The dosage of the active ingredients may vary depending on the route of administration, age and weight of the patient, nature and severity of the disorder to be treated and similar factors. The daily dose is 0.5-1000 mg, it being possible to give the dose as a single dose to be administered once or divided into two or more daily doses.

The present invention likewise relates to medicaments for the treatment of the abovementioned disorders, which comprise at least one quinoline derivative having the general formula (A), where the medicaments may where appropriate comprise suitable formulation substances and carriers.

Where no description is given for the preparation of the starting compounds, they are known to the skilled person or can be prepared in analogy to known compounds or to processes described herein. It is likewise possible to carry out all the reactions described herein in parallel reactors or using combinatorial operating techniques.

The mixtures of isomers can be fractionated by conventional methods such as, for example, crystallization, chromatography or salt formation into the enantiomers or E/Z isomers.

Salts are prepared in a conventional way by mixing a solution of the compound having the general formula (A) with the equivalent amount or an excess of a base or acid, which is in solution where appropriate, and removing the precipitate or working up the solution in a conventional way.

The present invention likewise relates to the process for preparing the quinoline derivatives according to the invention.

The intermediates preferably used for preparing the quinoline derivatives according to the invention having the general formula (A) are the following compounds having the general formulae (I) to (VII).

General description of the preparation of the compounds according to the invention:

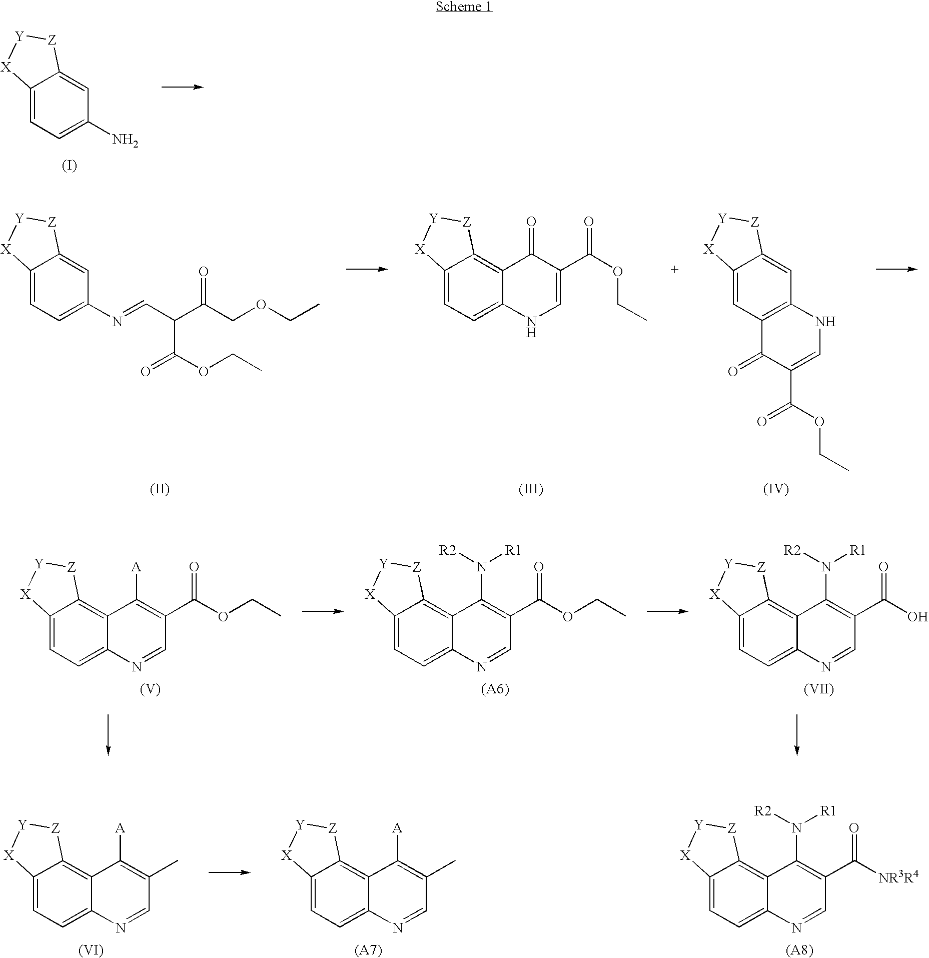

Quinoline derivatives according to the invention having the general formula (A) can be prepared for example by the route shown in scheme 1, in which the radical A can be for example halogen or —OS(O)₂CₙF₂ₙ₊₁ with n=1−3 and the radicals R1 and R2 may be as described in the claims, and the radicals W, X, Y and Z have the same meaning as in the general formula (A). The required starting materials are either commercially available or are prepared by processes disclosed in the literature, or in analogy to processes disclosed in the literature, or as described below.

Addition of ethyl di(ethoxymethylene)acetate onto a compound having the general formula (I) forms compounds having the general formula (II). These compounds are then cyclized, preferably under thermal conditions, to compounds having the general formula (III) (cf. Bioorg. Med. Chem. Lett. 2000, 10, 2815-2828). It is also possible to employ acids or Lewis acids in these cyclizations (cf. Monatsh. Chemie 1978, 109, 527.). The by-products of the general formula (IV) which may likewise be formed in this case can be removed at this stage.

Compounds having the general formula (V) are then prepared for example by reaction with thionyl chloride or phosphoryl chloride (for A=Cl) or perfluoroalkylsulphonic anhydrides (for A=perfluoroalkylsulphonyl) (cf. J. Med. Chem. 2005, 48, 1107-1131.) Compounds having the general formula (A6) can then be prepared from compounds of the general formula (V) by addition of amines. Coupling with the amines can take place under acidic, basic or neutral conditions, but also by transition metal-catalyzed coupling in the presence of suitable ligands (cf. Angew. Chemie 1998, 110, 2154-2177; Angew. Chemie 2000, 112, 4666-4668.).

Alternatively, it is possible to obtain compounds of the general formula (VI) from compounds of the general formula (V) by reduction (for example with lithium aluminium hydride; cf. J. Med. Chem. 1992, 35, 3413-3422). These can then in turn be converted into compounds of the general formula (A7) by addition of amines. Coupling with the amines can take place as described above under acidic, basic or neutral conditions, or else by transition metal-catalyzed coupling in the presence of suitable ligands (cf. Angew. Chemie 1998, 110, 2154-2177; Angew. Chemie 2000, 112, 4666-4668.).

Alternatively, the ester compounds of the general formula (A6) can be converted by hydrolysis into the free carboxylic acid (VII). Subsequent reaction with amines, for example using coupling reagents, then leads to the compounds of the general formula (A8).

The radicals X, Y and Z can where appropriate be further modified. Functional groups possibly present in the intermediates, such as carbonyl groups, hydroxy groups or amino groups, can be protected in the interim with protective groups by known processes.

An alternative preparation of compounds of the general formula (A6) starting from anthranilic acid derivatives is described for example in the literature (J. Med. Chem. 2001, 44, 822-833.).

It is alternatively possible before the reaction management described previously to prepare the final compounds according to the invention by parallel synthesis, for example in an automatic synthesizer.

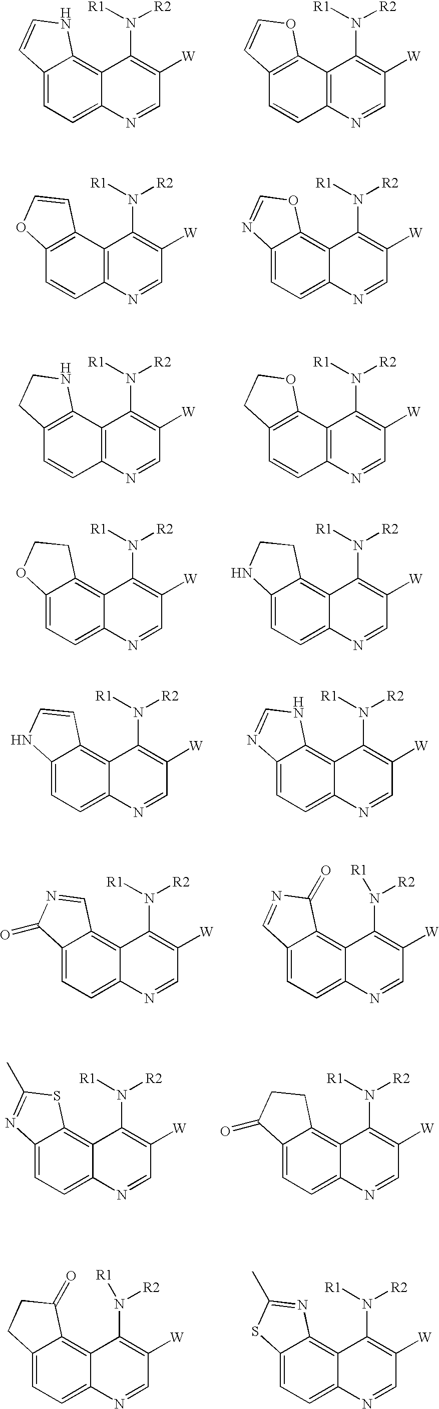

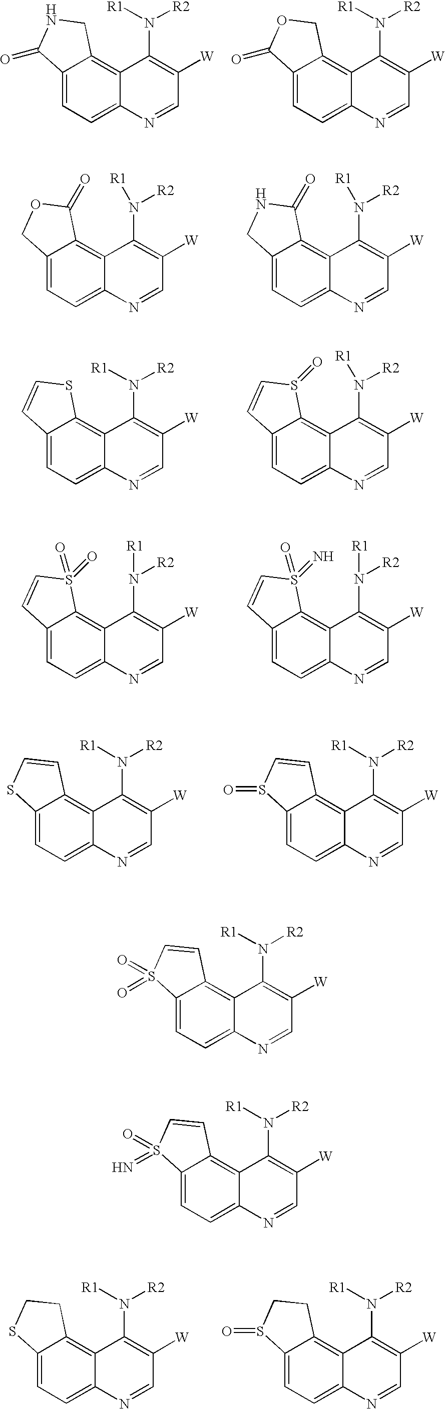

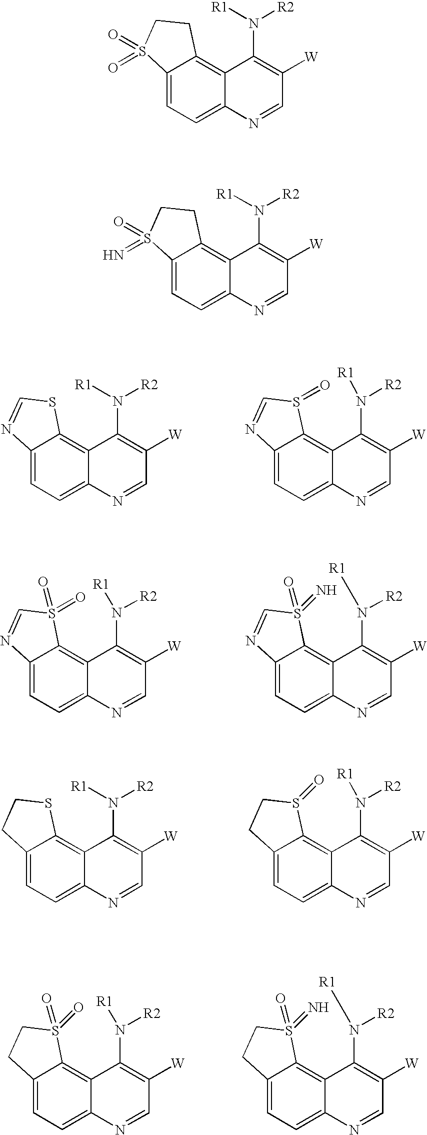

Experimental description of the preparation of the intermediates and of the products according to the invention of the general formula (A).

General Part

The naming of the chemical structures took place using the software tool Autonom 2000 for ISIS/Draw [MDL Information Systems Inc. (Elsevier MDL)].

### Preparation of 1,1-dioxo-2,3-dihydro-1H-1λ⁶-benzo[b]thiophen-5-ylamine

The final compound can be prepared in a five-stage sequence using literature methods starting from 2-chloro-5-nitrobenzaldehyde (J. Heterocyclic Chem. 2001, 38, 1025; J. Am. Chem. Soc. 1948, 70, 1957; Rec. Trav. Chim Pays-Bas 1954, 73, 819.) . . . ). An alternative possibility for converting the 5-nitro-benzo[b]thiophene into the sulphone is to convert the nitro compound into the benzo[b]thiophen-5-ylamine by reduction. This compound can then be converted into the target compounds in analogy to the sulphone.

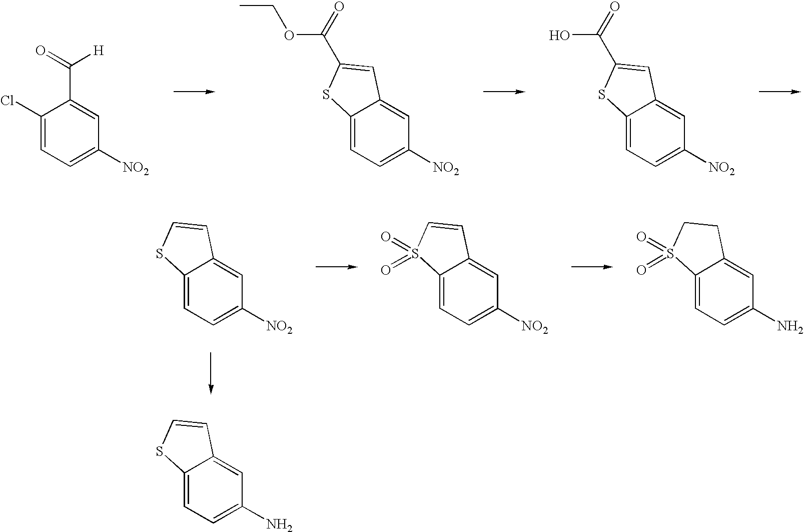

The amine 2 can also be assembled alternatively in accordance with precedence in the literature. 4-Nitrophenol is prepared from 4-chloronitrobenzene as described in J. Am. Chem. Soc. 1946, 68, 498-500. Starting therefrom it is possible to assemble the benzothiophene structure by cyclization in the presence of 2-bromoacetaldehyde diethyl acetal (cf. Bioorg. Med. Chem. Lett. 2004, 14, 5395-5399).

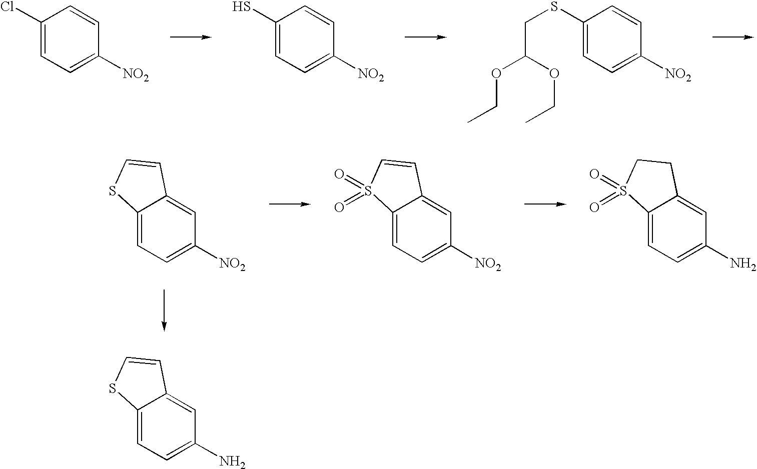

The amines generated in this way can then be converted by the reaction routes described in scheme 1 to compounds of the general formula (A).

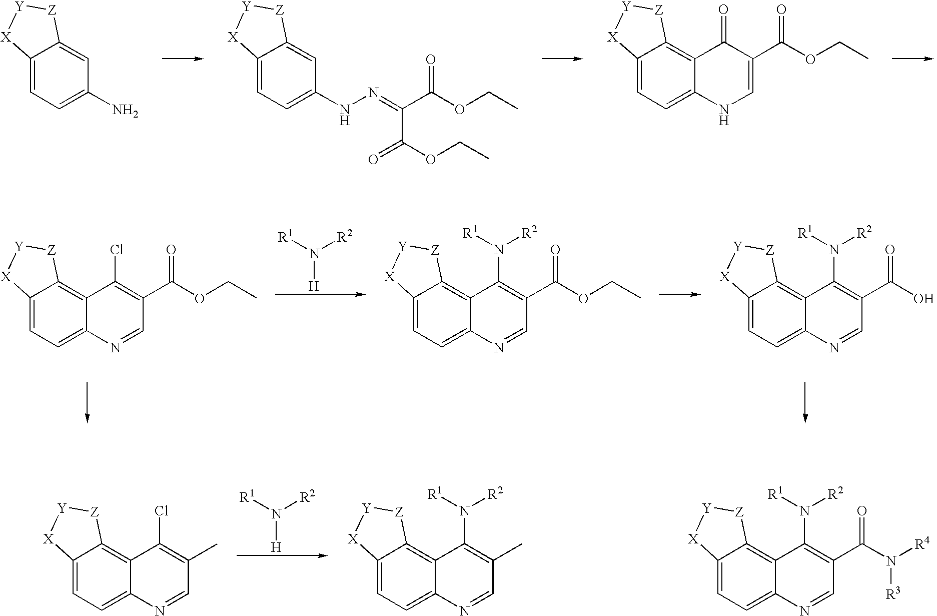

Amines listed for example in the following table can be introduced in accordance with the reaction scheme shown above in order to prepare the corresponding compounds of the general formula (A):

| Serial | |
|---|---|
| No. | Structure of amine |
| 1. | 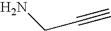 |
| 2. | 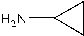 |
| 3. | 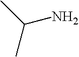 |
| 4. | 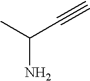 |
| 5. | 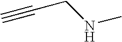 |
| 6. | 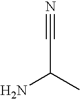 |
| 7. | 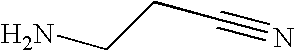 |
| 8. | 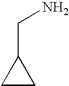 |
| 9. | 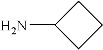 |
| 10. | 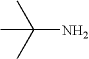 |
| 11. | 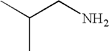 |
| 12. | 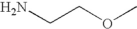 |
| 13. | 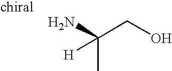 |
| 14. | 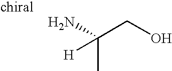 |
| 15. | 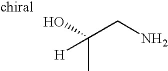 |
| 16. | 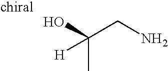 |
| 17. | 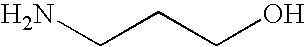 |
| 18. | 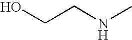 |
| 19. | 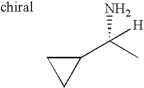 |
| 20. | 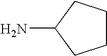 |
| 21. | 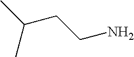 |
| 22. | 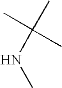 |
| 23. | 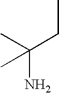 |
| 24. | 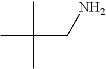 |
| 25. | 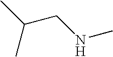 |
| 26. | 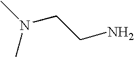 |
| 27. | 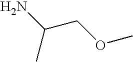 |
| 28. | 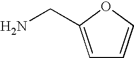 |
| 29. | 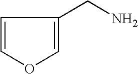 |
| 30. | 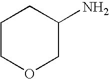 |
| 31. | 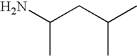 |
| 32. | 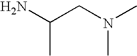 |
| 33. | 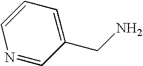 |
| 34. | 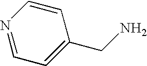 |
| 35. | 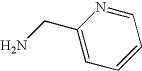 |
| 36. | 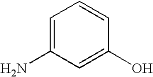 |
| 37. | 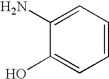 |
| 38. | 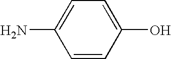 |
| 39. | 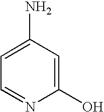 |
| 40. | 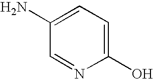 |
| 41. | 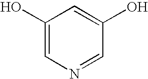 |
| 42. | 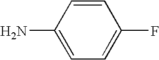 |
| 43. | 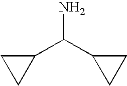 |
| 44. | 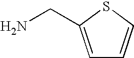 |
| 45. | 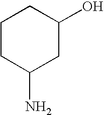 |
| 46. | 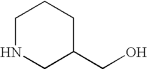 |
| 47. | 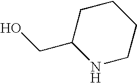 |
| 48. | 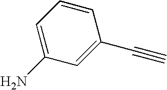 |
| 49. | 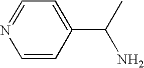 |
| 50. | 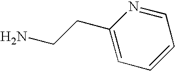 |
| 51. | 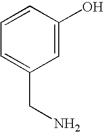 |
| 52. | 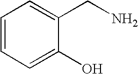 |
| 53. | 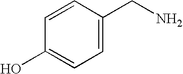 |
| 54. | 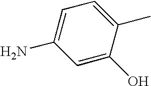 |
| 55. | 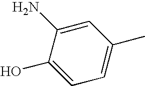 |
| 56. | 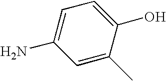 |
| 57. | 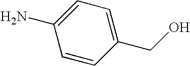 |
| 58. | 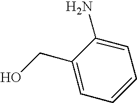 |
| 59. | 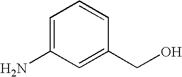 |
| 60. | 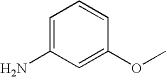 |
| 61. | 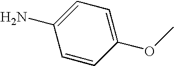 |
| 62. | 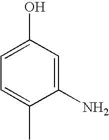 |
| 63. | 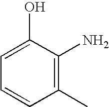 |
| 64. | 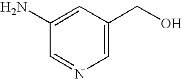 |
| 65. | 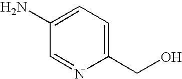 |
| 66. | 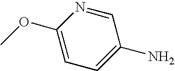 |
| 67. | 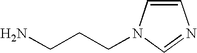 |
| 68. | 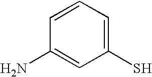 |
| 69. | 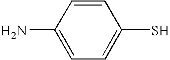 |
| 70. | 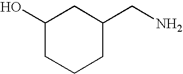 |
| 71. | 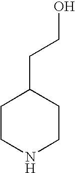 |
| 72. | 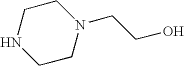 |
| 73 | 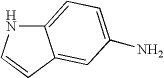 |
| 74. | 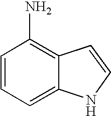 |
| 75. | 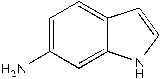 |
| 76. | 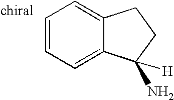 |
| 77. | 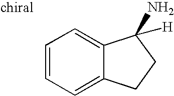 |
| 78. | 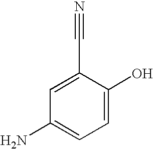 |
| 79. | 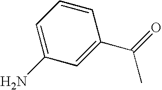 |
| 80. | 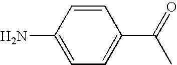 |
| 81. | 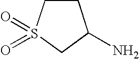 |
| 82. | 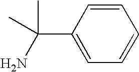 |
| 83. | 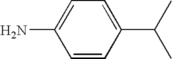 |
| 84. | 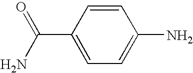 |
| 85. | 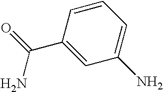 |
| 86. | 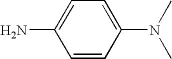 |
| 87. | 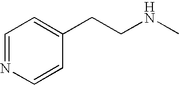 |
| 88. | 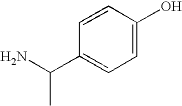 |
| 89. | 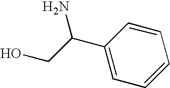 |
| 90. | 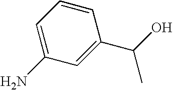 |
| 91. | 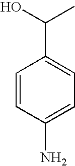 |
| 92. | 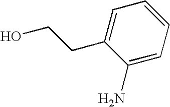 |
| 93. | 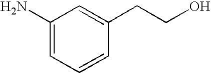 |
| 94. | 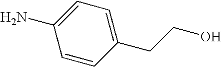 |
| 95. | 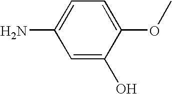 |
| 96. | 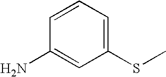 |
| 97. | 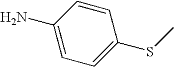 |
| 98. | 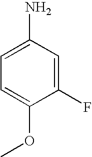 |
| 99. | 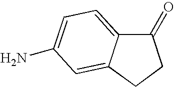 |
| 100. | 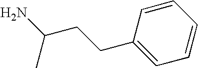 |
| 101. | 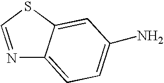 |
| 102. | 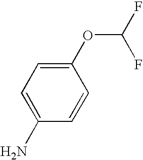 |
| 103. | 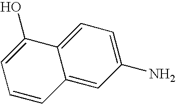 |
| 104. | 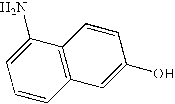 |
| 105. | 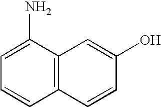 |
| 106. | 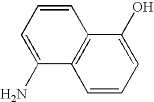 |
| 107. | 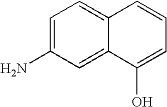 |
| 108. | 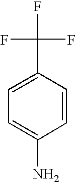 |
| 109. | 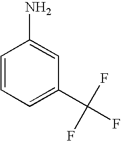 |
| 110. | 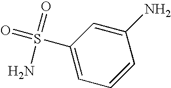 |
| 111. | 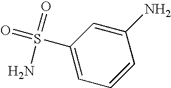 |
| 112. | 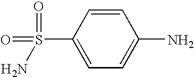 |
| 113. | 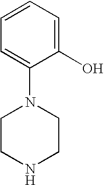 |
| 114. | 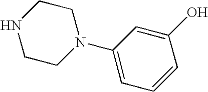 |
| 115. | 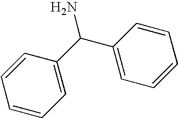 |

### Preparation of Compounds of Type (V)

### Preparation of diethyl 2-[(1,1-dioxo-2,3-dihydro-1H-1λ⁶-benzo[b]thiophen-5-ylamino)methylene]malonate

A solution of 340 mg of 1,1-dioxo-2,3-dihydro-1H-1λ⁶-benzo[b]thiophen-5-ylamine in 5 ml of diethyl ethoxymethylenemalonate is stirred at 130° C. for 1.5 hours. The reaction mixture is then diluted with ethyl acetate. It is washed with saturated aqueous sodium chloride solution, dried over sodium sulphate and concentrated in vacuo. The crude product is purified by column chromatography on silica gel with a hexane/ethyl acetate mixture. 613 mg of product are obtained.

¹H-NMR (d6-DMSO): δ=1.25 (6H); 3.32 (2H); 3.59 (2H); 4.18 (2H); 7.50 (1H); 7.54 (1H); 7.72 (1H); 8.42 (1H); 10.72 (1H).

### Preparation of 3,3,9-trioxo-2,3,6,9-tetrahydro-1H-3λ⁶-thieno[3,2-f]-quinoline-8-carboxylate

A solution of 100 mg of diethyl 2-[(1,1-dioxo-2,3-dihydro-1H-1λ⁶-benzo[b]thiophen-5-ylamino)methylene]malonate in 2 ml of diphenyl ether is stirred at 240° C. for 35 minutes. After cooling, cyclohexane is added, and the mixture is stirred at 23° C. for one hour. The precipitated product is filtered off with suction and recrystallized from a mixture of dichloromethane and methanol (95:5). 162 mg of product are obtained.

¹H-NMR (d6-DMSO): δ=1.28 (3H); 3.62 (2H); 3.96 (2H); 4.22 (2H); 7.72 (1H); 7.96 (1H); 8.56 (1H); 12.60 (1H).

### Preparation of ethyl 9-chloro-3,3-dioxo-2,3-dihydro-1H-3λ⁶-thieno[3,2-f]-quinoline-8-carboxylate

Ethyl 3,3,9-trioxo-2,3,6,9-tetrahydro-1H-3λ⁶-thieno[3,2-f]quinoline-8-carboxylate (300 mg, 0.98 mmol) is mixed with POCl₃ (0.55 ml, 5.86 mmol) and boiled under reflux for 5 h. The reaction mixture is added to ice-water and adjusted to pH 13 with 25% strength sodium hydroxide solution. The mixture is extracted with methylene chloride three times. The organic phase is dried over sodium sulphate. The residue (170 mg) after removal of the solvent is reacted further without purification.

### Preparation of diethyl 2-(benzo[b]thiophen-5-ylaminomethylene)malonate

A solution of 540 mg of benzo[b]thiophen-5-ylamine in 5 ml of diethyl ethoxymethylenemalonate is stirred at 130° C. for 1.5 hours. The reaction mixture is then diluted with ethyl acetate. It is washed with saturated aqueous sodium chloride solution, dried over sodium sulphate and concentrated in vacuo. The crude product is purified by column chromatography on silica gel with a hexane/ethyl acetate mixture. 1.88 g of product are obtained.

¹H-NMR (CDCl₃): δ=1.30-1.45 (6H); 4.20-4.38 (4H); 7.16 (1H); 7.30 (1H); 7.51 (1H); 7.58 (1H); 7.86 (1H); 8.60 (1H) 11.12 (1H).

### Preparation of ethyl 9-oxo-6,9-dihydrothieno[3,2-f]quinoline-8-carboxylate

A solution of 315 mg of diethyl 2-(benzo[b]thiophen-5-ylaminomethylene)-malonate in 2 ml of diphenyl ether is stirred at 240° C. for 35 minutes. After cooling, cyclohexane is added and the mixture is stirred at 23° C. for one hour. The precipitated product is filtered off with suction and recrystallized from a mixture of dichloromethane and methanol (95:5). 159 mg of product are obtained.

¹H-NMR (d6-DMSO): δ=1.30 (3H); 4.23 (2H); 7.61 (1H); 8.02 (1H); 8.34 (1H); 8.56 (1H); 8.94 (1H); 12.50 (1H).

### Preparation of ethyl 9-chlorothieno[3,2-f]quinoline-8-carboxylate

Ethyl 9-oxo-6,9-dihydro-thieno[3,2-f]quinoline-8-carboxylate (450 mg, 1.65 mmol) is mixed with POCl₃ (0.92 ml, 9.88 mmol) and boiled under reflux for 5 h. The reaction mixture is added to ice-water and adjusted to pH 13 with 25% strength sodium hydroxide solution. The mixture is extracted with methylene chloride three times. The organic phase is dried over sodium sulphate. The residue (400 mg) after removal of the solvent is reacted further without purification.

### General Procedure for Reacting ethyl 9-chloro-3,3-dioxo-2,3-dihydro-1H-3λ⁶-thieno[3,2-f]quinoline-8-carboxylate or ethyl 9-chlorothieno[3,2-f]quinoline-8-carboxylate with Various Amines (Gp 1)

Ethyl 9-chloro-3,3-dioxo-2,3-dihydro-1H-3λ⁶-thieno[3,2-f]quinoline-8-carboxylate or ethyl 9-chlorothieno[3,2-f]quinoline-8-carboxylate (1.0 eq.) is introduced into ethanol (86 eq.), mixed with amine (2.5 eq.) and then refluxed at a bath temperature of 90° C. for 1.5 h. After cooling, the crystals which have separated out are filtered off with suction and washed with ethanol. The mother liquor is evaporated to dryness, and the residue is purified by chromatography.

The following examples were prepared by the indicated procedure:

### EXAMPLE 1

### Ethyl 9-(2-hydroxyethylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (170 mg, 0.52 mmol) with 2-aminoethanol (0.078 ml, 1.3 mmol) by GP 1 results, after purification by chromatography (silica gel, ethyl acetate/n-hexane with ethyl acetate: 0-100%), in the desired product in 25% yield (47 mg).

¹HNMR (300 MHz, DMSO): δ 1.32 (t, 3H), 3.21-3.24 (m, 2H), 3.46-3.50 (m, 2H), 3.64 (t, 2H), 3.87 (t, 2H), 4.32 (q, 2H), 4.87 (t, 1H), 7.53 (t, 1H), 7.89 (s, 2H), 8.83 (s, 1H).

### EXAMPLE 2

### Ethyl 9-(3-hydroxypropylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (150 mg, 0.46 mmol) with 3-aminopropan-1-ol (87 mg, 1.15 mmol) by GP 1 results, after purification by chromatography (silica gel, ethyl acetate/n-hexane with ethyl acetate: 0-100%, then ethyl acetate/methanol with methanol: 0-30%), in the desired product in 25% yield (43 mg).

¹HNMR (300 MHz, DMSO): δ 1.36 (t, 3H), 1.72-1.74 (m, 2H), 3.37-3.29 (m, 2H), 3.47 (q, 2H), 3.68 (t, 2H), 3.92 (t, 2H), 4.37 (q, 2H), 4.72-4.76 (m, 1H), 7.48 (t, 1H), 7.93 (s, 2H), 8.85 (s, 1H).

### EXAMPLE 3

### Ethyl 9-(2-dimethylaminoethylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (200 mg, 0.61 mmol) with N,N-dimethylethylenediamine (0.17 ml, 1.53 mmol) by GP 1 results, after purification by chromatography (silica gel, ethyl acetate/n-hexane with ethyl acetate: 0-100%), in the desired product in 48% yield (110 mg).

¹HNMR (300 MHz, DMSO): δ 1.31 (t, 3H), 2.14 (s, 6H), 3.27-3.30 (m, 2H), 3.15 (br, 2H), 3.67 (t, 2H), 3.88 (t, 2H), 4.31 (q, 2H), 7.46 (br, 1H), 7.88 (s, 2H), 8.80 (s, 1H).

### EXAMPLE 4

### Ethyl 9-(2-acetylaminoethylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (150 mg, 0.46 mmol) with N-acetylethylenediamine (118 mg, 1.15 mmol)) by GP 1 results, after purification by chromatography (silica gel, ethyl acetate/n-hexane with ethyl acetate 0-100%, then ethyl acetate/methanol with methanol: 0-30%), in the desired product in 55% yield (89 mg).

¹HNMR (300 MHz, DMSO): δ 1.32 (t, 3H), 1.62 (s, 3H), 3.19-3.24 (m, 4H), 3.63 (t, 2H), 3.87 (t, 2H), 4.31 (q, 2H), 7.42 (t, 1H), 7.87-7.89 (m, 3H), 8.82 (s, 1H).

### EXAMPLE 5

### Ethyl 9-(2-morpholin-4-ylethylamino)-3,3-dioxo-2,3-dihydro-1H -3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (150 mg, 0.461 mmol) with 2-morpholin-4-ylethylamine (150 mg, 1.15 mmol) by GP 1 results, after purification by chromatography (silica gel, ethyl acetate/n-hexane with ethyl acetate: 0-100%, then ethyl acetate/methanol with methanol: 0-10%), in the desired product in 52% yield (100 mg).

¹HNMR (400 MHz, DMSO): δ 1.31 (t, 3H), 2.30 (br, 4H), 3.19 (br, 2H), 3.47 (br, 4H), 3.70 (t, 2H), 3.95 (t, 2H), 4.31 (q, 2H), 7.48 (br, 1H), 7.89 (s, 2H), 8.81 (s, 1H).

### EXAMPLE 6

### Ethyl 9-cyclopropylamino-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (200 mg, 0.61 mmol) with cyclopropylamine (88 mg, 1.53 mmol) by GP 1 results, after purification by chromatography (silica gel, ethyl acetate/n-hexane with ethyl acetate: 0-100%), in the desired product in 52% yield (110 mg).

¹HNMR (400 MHz, DMSO): δ 0.52-0.54 (m, 2H), 0.67-0.70 (m, 2H), 1.31 (t, 3H), 2.78-2.80 (m, 1H), 3.61 (t, 2H), 3.96 (t, 2H), 4.30 (q, 2H), 7.07 (s, 1H), 7.86 (s, 2H), 8.68 (s, 1H).

### EXAMPLE 7

### Ethyl 9-isopropylamino-3,3-dioxo-2,3-dihydro-1H-3λ⁶-thieno[3,2-f]-quinoline-8-carboxylate

Reaction of ethyl 9-chloro-3,3-dioxo-2,3-dihydro-1H-3λ⁶-thieno[3,2-f]-quinoline-8-carboxylate (200 mg, 0.61 mmol) with isopropylamine (0.13 ml, 1.53 mmol) by GP 1 results, after purification by chromatography (silica gel, ethyl acetate/n-hexane with ethyl acetate: 0-100%), in the desired product in 45% yield (97 mg).

¹HNMR (400 MHz, DMSO): δ 1.08 (d, 6H), 1.32 (t, 3H), 3.62-3.67 (m, 3H), 3.87 (t, 2H), 4.35 (q, 2H), 7.14 (d, 1H), 7.93 (s, 2H), 8.92 (s, 1H).

### EXAMPLE 8

### Ethyl 9-benzylamino-3,3-dioxo-2,3-dihydro-1H-3λ⁶-thieno[3,2-f]quinoline-8-carboxylate

Ethyl 9-chloro-3,3-dioxo-2,3-dihydro-1H-3λ⁶-thieno[3,2-f]-quinoline-8-carboxylate (200 mg, 0.61 mmol) is reacted with benzylamine (0.17 ml, 1.53 mmol) by GP 1. The resulting crystals are filtered off with suction and washed with ethanol. The desired product is obtained in 36% yield (87 mg).

¹HNMR (400 MHz, DMSO): δ 1.16 (t, 3H), 3.66 (t, 2H), 3.96 (t, 2H), 4.15 (q, 2H), 4.49 (d, 2H), 7.08 (d, 2H), 7.18-7.26 (m, 3H), 7.71 (t, 1H), 7.93 (s, 2H), 8.77 (s, 1H).

### EXAMPLE 9

### Ethyl 3,3-dioxo-9-phenylamino-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (200 mg, 0.61 mmol) with aniline (0.14 ml, 1.53 mmol) by GP 1 results, after purification by chromatography (silica gel, ethyl acetate/n-hexane with ethyl acetate: 0-100%), in the desired product in 47% yield (110 mg).

¹HNMR (400 MHz, DMSO): δ 1.12 (t, 3H), 3.50-3.59 (m, 4H), 4.05 (q, 2H), 6.80 (d, 2H), 6.91 (t, 1H), 7.19 (t, 2H), 7.99 (d, 1H), 8.08 (d, 1H), 9.06 (s, 1H), 9.23 (s, 1H).

### EXAMPLE 10

### Ethyl 9-(4-methoxyphenylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (150 mg, 0.46 mmol) with 4-methoxyphenylamine (142 mg, 1.15 mmol) by GP 1 results, after purification by chromatography (silica gel, ethyl acetate/n-hexane with ethyl acetate: 0-100%, then ethyl acetate/methanol with methanol: 0-10%), in the desired product in 25% yield (48 mg).

¹HNMR (400 MHz, DMSO): δ 1.20 (t, 3H), 3.46 (br, 4H), 3.65 (s, 3H), 4.12 (q, 2H), 6.79 (s, 4H), 7.95 (d, 1H), 8.02 (d, 1H), 9.04 (s, 1H), 9.36 (s, 1H).

### EXAMPLE 11

### Ethyl 9-(4-hydroxy-phenylamino)-3,3-dioxo-2,3-dihydro-1H-3λ⁶-thieno[3,2-f]quinoline-8-carboxylate

Reaction of ethyl 9-chloro-3,3-dioxo-2,3-dihydro-1H-3λ⁶-thieno[3,2-f]-quinoline-8-carboxylate (150 mg, 0.46 mmol) with 4-aminophenol (125 mg, 1.15 mmol) by GP 1 results, after purification by chromatography (silica gel, ethyl acetate/n-hexane with ethyl acetate: 0-100%, then ethyl acetate/methanol with methanol: 0-10%), in the desired product in 22% yield (40 mg).

¹HNMR (400 MHz, DMSO): δ 1.23 (t, 3H), 3.36 (br, 2H), 3.46 (t, 2H), 4.13-4.19 (m, 2H), 6.61 (d, 2H), 6.69 (d, 2H), 7.94 (d, 1H), 8.00 (d, 1H), 9.02 (s, 1H), 9.26 (s, 1H), 9.47 (s, 1H).

### EXAMPLE 12

### Ethyl 9-phenylaminothieno[3,2-f]quinoline-8-carboxylate

Reaction of ethyl 9-chlorothieno[3,2-f]quinoline-8-carboxylate (200 mg, 0.69 mmol) with aniline (0.16 ml, 1.713 mmol) by GP 1 results, after purification by chromatography (silica gel, ethyl acetate/n-hexane with ethyl acetate: 0-100%), in the desired product in 28% yield (67 mg).

¹HNMR (400 MHz, DMSO): δ 1.25 (t, 3H), 4.28 (q, 2H), 6.63 (d, 2H), 6.80 (t, 1H), 7.06 (t, 2H), 7.73 (d, 1H), 7.86 (dd, 1H), 7.92 (d, 1H), 8.41 (d, 1H), 9.17 (s, 1H), 9.59 (s, 1H).

### EXAMPLE 13

### Ethyl 9-benzylaminothieno[3,2-f]quinoline-8-carboxylate

Reaction of ethyl 9-chlorothieno[3,2-f]quinoline-8-carboxylate (200 mg, 0.69 mmol) with benzylamine (0.19 ml, 1.71 mmol) by GP 1 results, after purification by chromatography (silica gel, ethyl acetate/n-hexane with ethyl acetate: 0-100%), in the desired product in 13% yield (32 mg).

¹HNMR (400 MHz, DMSO): δ 1.25 (t, 3H), 4.26 (q, 2H), 4.35 (d, 2H), 7.00 (d, 2H), 7.18-7.24 (m, 3H), 7.82 (d, 1H), 8.06 (d, 1H), 8.15 (t, 1H), 8.25-8.27 (m, 1H), 8.38 (d, 1H), 8.97 (s, 1H)

Biological Tests on the Compounds

Test System for EphB4

A mixture of 20 ng/ml recombinanter EphB4 kinase (ProQinase GmbH, Freiburg, Germany), 2.67 μg/ml polyGluAlaTyr, 2 μM ATP, 25 mM HEPES (pH 7.3), 5 mM MgCl₂, 1 mM MnCl₂, 2 mM DTT, 0.1 mM NaVO₄, 1% (v/v) glycerol, 0.02% NP40, EDTA-free protease inhibitors (Complete from Roche, 1 tablet in 50 ml) is incubated at 20° C. for 10 min. Test substances are dissolved in 100% DMSO and introduced in 0.017 times the volume before the start of the reaction. 60 minutes after addition of 1.7 times the volume of a solution of 50 mM Hepes pH 7.0, 0.2% BSA, 0.14 μg/ml PT66-Europium, 3.84 μg/ml SA-XL665, 75 mM EDTA, the mixture is measured in a Perkin-Elmer Discovery HTRF measuring instrument.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding 10 2006 02 9446.7 application No., filed Jun. 21, 2006, and U.S. Provisional Application Ser. No. 60/816,630, filed Jun. 27, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Quinoline derivative having the general formula (A): 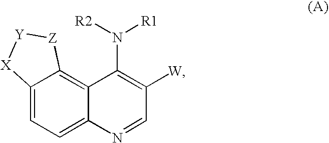
where
W is equal to methyl, C(O)OR⁴, C(O)NR³R⁴;
R¹ and R² are identical or different and are selected independently of one another from the group comprising hydrogen, hydroxy, halogen, nitro, cyano, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚPO₃(R⁶)₂, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵, or —OR⁵, where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally form together a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
the N-oxides, solvates, hydrates, stereoisomers, diastereomers, enantiomers and salts thereof.

2. Quinoline derivative of the general formula (A) according to claim 1, where:
W is equal to methyl, C(O)OR⁴, C(O)NR³R⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
the N-oxides, solvates, hydrates, stereoisomers, diastereomers, enantiomers and salts thereof.

3. Quinoline derivative of the general formula (A) according to claim 1, where:
W is equal to C(O)OR⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
X, Y, Z are identical or different and are selected independently of one another from the group comprising —CR³═, —CR³R⁴—, —C(O)—, —N═, —S—, —O—, —NR³—, —S(O)₂—, —S(O)— and —S(O)(N═R³)—, and single or double bonds are present between X, Y and Z, but a maximum of two of the three radicals X, Y and Z are identical with —CR³═, —CR³R⁴—;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
the N-oxides, solvates, hydrates, stereoisomers, diastereomers, enantiomers and salts thereof.

4. Quinoline derivatives according to claim 1, of the general formulae (A1-A5): 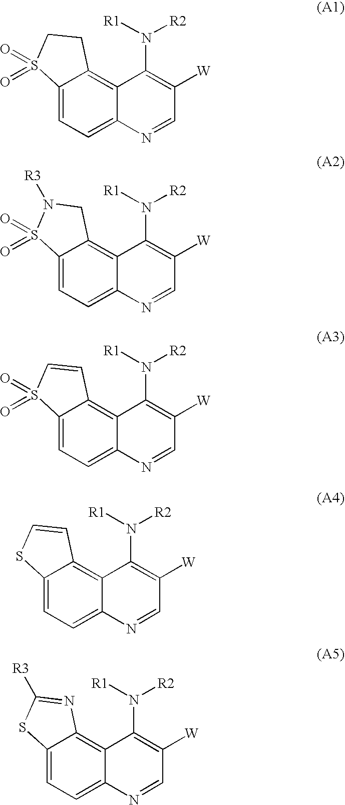
where:
W is equal to methyl, C(O)OR³, C(O)NR³R⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
R³ and R⁴ are selected independently of one another from the group comprising hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁶, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
the N-oxides, solvates, hydrates, stereoisomers, diastereomers, enantiomers and salts thereof.

5. Quinoline derivatives according to claim 1, of the general formulae (A1-A5): 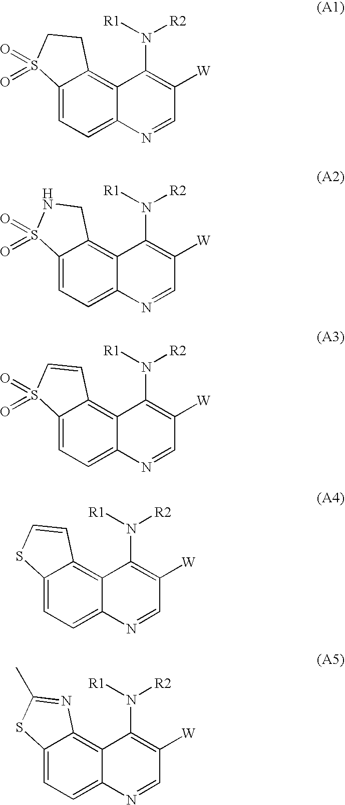
where:
W is equal to methyl, C(O)OR⁴, C(O)NHR⁴;
R¹ and R² are identical or different and are selected independently of one another from the group comprising hydrogen, —C₁-C₆-alkyl, —C₁-C₄-hydroxyalkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —CHR⁵R⁶, —(CH₂)ₚ—SR⁵ and —CR⁵(OH)—R⁶, where —C₁-C₆-alkyl, —C₂-C₆-alkenyl, —C₂-C₆-alkynyl, —C₃-C₁₀-cycloalkyl, —C₃-C₁₂-heterocycloalkyl, —C₆-C₁₂-aryl, —C₅-C₁₈-heteroaryl or —C₁-C₆-alkoxy are unsubstituted or are substituted one or more times independently of one another by hydroxy, halogen, nitro, cyano, —NR⁵R⁶, —C(O)NR⁵R⁶, —S(O)₂NR⁵R⁶, —NR⁵S(O)₂R⁶, —NR⁵C(O)R⁶, —SR⁵, —R⁵ or —OR⁵ where the carbon framework of the —C₃-C₁₀-cycloalkyl and of the —C₁-C₁₀-alkyl may comprise one or more times independently of one another nitrogen, oxygen, sulphur atoms, —NR⁴ or C═O groups or one or more double bonds, or R¹ and R² optionally together form a bridge of 3-10 methylene units, where up to two methylene units are optionally replaced by O, S or —NR⁴, and where the phenyl radical is optionally substituted one or more times independently of one another by hydroxy, halogen, nitro, cyano, phenyl, —NR⁵R⁶, alkyl or —OR⁵;
R⁴ is hydrogen, —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl, where —C₁-C₁₀-alkyl, —C₂-C₆-alkenyl, —C₂-C₆-alkynyl, —C₃-C₁₀-cycloalkyl, —C₃-C₁₂-heterocycloalkyl or —C₁-C₁₀-alkanoyl is unsubstituted or is substituted one or more times independently of one another by hydroxy, halogen, nitro, cyano, phenyl, —NR⁵R⁵, alkyl, —SR⁵ or —OR⁵,
R⁵ and R⁶ are identical or different and are selected independently of one another from the group comprising hydrogen, —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, where —C₁-C₁₀-alkyl, —C₂-C₁₀-alkenyl, —C₂-C₁₀-alkynyl, —C₁-C₆-alkoxy, —C₃-C₁₀-cycloalkyl, —C₃-C₁₂-heterocycloalkyl, —C₆-C₁₂-aryl or —C₅-C₁₈-heteroaryl are unsubstituted or are substituted one or more times independently of one another by hydroxy, halogen, cyano, nitro, —OR⁷, —NR⁷R⁸, —C(O)NR⁷R⁸, —C(O)OR⁷ or —C₁-C₆-alkyl, where —C₁-C₆-alkyl is unsubstituted or is substituted one or more times independently of one another by halogen, hydroxy, cyano, —NR⁷R⁸, —OR⁷ or phenyl; or R⁵ and R⁶ optionally together form a bridge of 3-10 methylene units, where up to two methylene units are optionally replaced by O, S or NR⁴;
R⁷, R⁸ are identical or different and are selected independently of one another from the group comprising hydrogen, —C₁-C₄-alkyl, —C₆-C₁₂-aryl and —C₅-C₁₈-heteroaryl, where alkyl, aryl, heteroaryl is unsubstituted or is substituted one or more times independently of one another by halogen or alkoxy, or R⁷ and R⁸ optionally together form a bridge of 3-10 methylene units, where up to two methylene units are optionally replaced by O, S or —NR⁴;
m′, m″=independently of one another 0, 1, 2, 3, or 4,
n=1, 2, 3, 4, 5, or 6,
p=0, 1, 2, 3, 4, 5, or 6, and
the N-oxides, solvates, hydrates, stereoisomers, diastereomers, enantiomers and salts thereof.

6. Quinoline derivatives according to claim 1, which are selected from:
ethyl 9-(2-hydroxyethylamino)-3,3-dioxo-2,3-dihydro-1H-3λ⁶-thieno[3,2-f]quinoline-8-carboxylate;
ethyl 9-(3-hydroxypropylamino)-3,3-dioxo-2,3-dihydro-1H-3λ⁶-thieno[3,2-f]quinoline-8-carboxylate;
ethyl 9-(2-dimethylaminoethylamino)-3,3-dioxo-2,3-dihydro-1H-3λ⁶-thieno[3,2-f]quinoline-8-carboxylate;
ethyl 9-(2-acetylaminoethylamino)-3,3-dioxo-2,3-dihydro-1H-3λ⁶-thieno[3,2-f]quinoline-8-carboxylate;
ethyl 9-(2-morpholin-4-ylethylamino)-3,3-dioxo-2,3-dihydro-1H-3λ⁶-thieno[3,2-f]-quinoline-8-carboxylate;
ethyl 9-cyclopropylamino-3,3-dioxo-2,3-dihydro-1H-3λ⁶-thieno[3,2-f]quinoline-8-carboxylate;
ethyl 9-isopropylamino-3,3-dioxo-2,3-dihydro-1H-3λ⁶-thieno[3,2-f]quinoline-8-carboxylate;
ethyl 9-benzylamino-3,3-dioxo-2,3-dihydro-1H-3λ⁶-thieno[3,2-f]quinoline-8-carboxylate;
ethyl 3,3-dioxo-9-phenylamino-2,3-dihydro-1H-3λ⁶-thieno[3,2-f]quinoline-8-carboxylate;
ethyl 9-(4-methoxyphenylamino)-3,3-dioxo-2,3-dihydro-1H-3λ⁶-thieno[3,2-f]-quinoline-8-carboxylate;
ethyl 9-(4-hydroxyphenylamino)-3,3-dioxo-2,3-dihydro-1H-3λ⁶-thieno[3,2-f]-quinoline-8-carboxylate;
ethyl 9-phenylaminothieno[3,2-f]quinoline-8-carboxylate;
ethyl 9-benzylaminothieno[3,2-f]quinoline-8-carboxylate.

7. Process for preparing the quinoline derivative according to claim 1, where an intermediate of the general formula V: 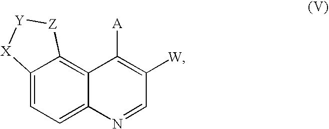
in which W, X, Y and Z are as defined in claim 1, and A is a leaving group,
is reacted with a reagent of the general formula V′: 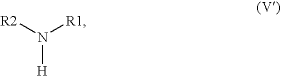
in which R1 and R2 are as defined in claim 1,
to give a compound of the general formula (A): 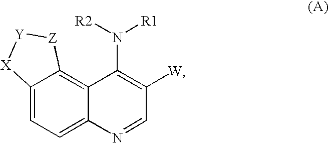
in which W, X, Y, Z, R1 and R2 are as defined.

8. Use of the quinoline derivative according to claim 1, for producing a medicament.

9. Use of the quinoline derivative according to claim 1, for producing a medicament for the treatment of disorders in which angiogenesis, lymphangiogenesis or vasculogenesis or a disorder of the blood vessels, or a disorder caused by hyperproliferation of body cells, or a chronic or acute neurodegenerative disorder.

10. Use of the quinoline derivative according to claim 1, for diagnostic purposes in vitro or in vivo for identifying receptors in tissues by means of autoradiography or PET.

11. Use of the quinoline derivative according to claim 1, as inhibitor of Eph receptor kinases.

12. Use of the quinoline derivative according to to claim 1, in the form of a pharmaceutical product for enteral, parenteral and oral administration.

13. Medicaments which comprise at least one quinoline derivative according to claim 1, as suitable formulation substances and carriers.

14. A method of treating angiogenesis, lymphangiogenesis or asculogenesis or a disorder of the blood vessels, or a disorder caused by hyperproliferation of body cells, or a chronic or acute neurodegenerative disorder, comprising administering a compound of claim 1.
